(19) United States
(12) Patent Application Publication
(10) Pub. No.: US 2021/0077526 A1
(43) Pub. Date: Mar. 18, 2021

(54) SUB-NANOMETER GOLD STICKER AND METHODS FOR PROTECTING AGAINST ENDOTOXIN-INDUCED SEPSIS THEREOF

(71) Applicant: NATIONAL HEALTH RESEARCH INSTITUTES, Miaoli County (TW)

(72) Inventor: Shu Yi LIN, Miaoli County (TW)

(21) Appl. No.: 17/041,653

(22) PCT Filed: Mar. 27, 2018
(86) PCT No.: PCT/US2018/024681

§ 371 Date: Sep. 25, 2020

(51) Int. Cl. A61K 33/242; C09D 179/02

(52) U.S. Cl. CPC A61K 33/242; B82Y 5/00; C09D 179/02

(57) ABSTRACT

A sub-nanometer gold sticker for blocking efficiently endotoxin activity to protect against sepsis is disclosed. The sub-nanometer gold sticker comprises a gold nanocluster that serves as a flake-like substrate and a coating of short alkyl motifs that act as an adhesive, allowing the sub-nanometer gold sticker to dock with LPS by compacting the intramolecular hydrocarbon chain-chain distance (d-spacing) of lipid A, an endotoxicity active site that can cause overwhelming cytokine induction resulting in sepsis progression. Methods of blocking endotoxin activity, and suppressing pro-inflammatory cytokines are also disclosed. Also disclosed is a method of protecting against endotoxin-induced sepsis via increasing critical micelle concentration for the inhibition of LPS non-lamellar aggregation.

### BACKGROUND OF THE INVENTION

### Field of the Invention

The present invention relates to a sticker, and more specifically to sub-nanometer gold sticker for protecting against endotoxin-induced sepsis. The present invention also relates to methods for protecting against endotoxin-induced sepsis by compacting lipid A via the sub-nanometer gold sticker.

### Description of the Prior Arts

In spite of endotoxicity constituting a challenging clinical problem, no drugs or therapeutic strategies that can successfully address this issue have been identified yet. The dangerous biological outcomes of endotoxicity, including excessive inflammation and even impaired immunity that can potentially lead to fatal sepsis and shock, are understood to be strongly associated with the molecular conformation of lipid A of lipopolysaccharide (LPS) due to its influence on the binding affinity of the natural host-guest interaction between the endotoxin (i.e., LPS) and the toll-like receptor 4 (TLR4)-MD2 complex. Since LPS is an amphiphilic molecule that can spontaneously self-assemble to form various aggregates under physiological conditions, different aggregate types can change the molecular conformation of lipid A by fine-tuning the intramolecular hydrocarbon chain-chain distance (d-spacing) of individual LPS molecules. In general, the conformation of lipid A is simplified for descriptive purposes as consisting of cylindrical and conical shapes, which can be derived from two typical aggregates of LPS, such as lamellar and non-lamellar aggregates. The term “cylindrical and conical shapes” is used because the d-spacing distance of individual lipid A domains is either almost equal to or greater than the cross-section of the disaccharide backbone (a portion of an LPS molecule) that acts as a linker to bundle several hydrocarbon chains of lipid A. The conformation of lipid A with a conical shape, in which the d-spacing represents a looser packing density, is able to activate the host-guest complex between LPS and the TLR4-MD2 complex for cytokine induction. Moreover, the strength profile of cytokine can be dramatically enhanced when the LPS aggregation becomes a cubic type, wherein the lipid A has one of the loosest packing densities. In contrast, the compact packing density between intramolecular hydrocarbon chains seen in lamellar LPS aggregates can result in the reduction or even elimination of cytokine induction. In biological environments, however, the conformation of lipid A is prone to form the looser packing density due to the excellent stability of the cubic aggregate.

### SUMMARY OF THE INVENTION

Endotoxicity originating from a dangerous debris (i.e., lipopolysaccharide, LPS) of gram-negative bacteria is a challenging clinical problem, but no drugs or therapeutic strategies that can successfully address this issue have been identified yet.

To overcome the shortcomings of above said, the objective of the present invention is to provide a sub-nanometer gold sticker that can efficiently block endotoxin activity to protect against sepsis.

In one aspect, the present invention relates to a sub-nanometer gold sticker, wherein the sub-nanometer gold sticker is comprising gold nanoparticles encapsulated within a polyamidoamine (PAMAM) dendrimer and a coating of alkyl motif.

In one embodiment of the invention, the gold nanoparticles are encapsulated within a dendrimer to form a gold nanoparticle-dendrimer complex.

In another embodiment of the present invention, the shape of the gold nanoparticle-dendrimer complex is about flake-like structure.

In another embodiment of the present invention, the PAMAM dendrimer has branched amines or branched hydroxyl groups.

In another embodiment of the present invention, wherein the PAMAM dendrimer includes, but is not limited to PAMAM generation 1 (G1), PAMAM generation 2 (G2), PAMAM generation 3 (G3), PAMAM generation 4 (G4), PAMAM generation 5 (G5), PAMAM generation 6 (G6), PAMAM generation 7 (G7), PAMAM generation 8 (G8), PAMAM generation 9 (G9), and PAMAM generation 10 (G10) dendrimers.

In another embodiment of the present invention, the PAMAM dendrimer is GₙNH₂ dendrimer or GₙOH dendrimer, wherein n is 0 to 4.

In another embodiment of the present invention, the PAMAM dendrimer is a generation 4 (G4) dendrimer. Preferably, the PAMAM dendrimer is G₄NH₂ dendrimer or G₄OH dendrimer.

In another embodiment of the present invention, the alky motif includes, but is not limited to methyl groups or ethyl groups.

In another embodiment of the present invention, the neighbor distance of gold atoms of gold nanoparticles ranges from 0.285 nm to 0.289 nm.

In one embodiment of the present invention, the sub-nanometer stickers allowing the sticker to dock with LPS by compacting the intramolecular hydrocarbon chain-chain distance (d-spacing) of lipid A, an endotoxicity active site that can cause overwhelming cytokine induction resulting in sepsis progression.

In another embodiment of the present invention, the d-spacing values of lipid A is decreasing from 4.19 Å to either 3.85 Å or 3.54 Å, indicating more dense packing densities in the presence of sub-nanometer gold stickers.

In another embodiment of the present invention, the sub-nanometer stickers resulting in more dense packing densities, in addition to increasing the CMC, and that might be protecting against sepsis.

In another embodiment of the present invention, the concentrations of key pro-inflammatory NF-κB-dependent cytokines, including, but not limited to plasma TNF-α, IL-6 and IL-1β, and CXC chemokines, in LPS-challenged mice showed a noticeable decrease.

In another aspect, the invention relates to a method of suppressing pro-inflammatory cytokines, comprising steps of administering to a LPS-infected mammal in need thereof an effective amount of sub-nanometer stickers.

In another embodiment of the present invention, the pro-inflammatory cytokines include, are not limited to NF-κB, TNF-α, IL-6 and IL-1β, CXC chemokine profiles, IL-12p40, GM-CSF or GRPα (KC).

In another embodiment of the present invention, wherein said effective amount of sub-nanometer stickers is dependent on the molar ratio of sub-nanometer stickers to LPS. Preferably, the molar ratio of sub-nanometer stickers to LPS is 1:2. According to the present invention, the effective amount of sub-nanometer stickers is about 50˜100 mg/kg body weight, which is dependent on the amount of LPS. Preferably, said effective amount of sub-nanometer stickers concentration of about 75 mg/kg body weight.

In another aspect of the present invention, the invention relates to a method of prolonging the survival time significantly in LPS-induced sepsis. The sub-nanometer gold stickers of the present invention could target lipid A of LPS to deactivate endotoxicity by compacting its packing density, which might constitute a potential therapeutic strategy for the early prevention of sepsis caused by gram-negative bacterial infection.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 illustrates a simple model representing the possible correlation between the packing density of lipid A of LPS and sepsis progression, wherein the rectangle indicates the formation of a sub-nanometer gold sticker inside a dendrimer, in which steps 1 and 2 include the synthesis and alkyl-motif modification of gold nanoclusters, respectively. The conformation of lipid A is depicted as being conical (left side) and then cylindrical (right side) in shape to correlate with the change of CMC as well as the difference in endotoxicity.

FIG. 2A shows the photoluminescence spectra and photograph showing the size of the SAuNCs to be less than 1 nm.

FIG. 2B shows HRTEM images and ED patterns (inset images) of SAuNCs indicating that the gold atoms can self-stack to form thin-films and alignments with different orientations. White arrows show the distances between individual gold atoms.

FIG. 2C shows HRTEM images and ED patterns (inset images) of SAuNCs indicating that the gold atoms can self-stack to form thin-films and alignments with different orientations. White arrows show the distances between individual gold atoms.

FIG. 2D shows HRTEM images and ED patterns (inset images) of SAuNCs indicating that the gold atoms can self-stack to form thin-films and alignments with different orientations. White arrows show the distances between individual gold atoms.

FIG. 3 shows ¹H NMR spectra of sticker-A, sticker-M, and sticker-E, respectively. The peaks of methyl and ethyl were appeared at approximately 2.5˜4 ppm as well.

FIG. 4A shows the comparisons of SAuNCs self-stacking before.

FIG. 4B shows the comparisons of SAuNCs self-stacking after the decoration of alkyl motifs on copper grids. Inset images show ED patterns; the undecorated gold atoms can easily self-stack to form thin-films and a well-ordered alignment compared with the decorated atoms. The white arrows show the distances between individual gold atoms.

FIG. 5A shows the measurement of CMC and d-spacing for LPS in the absence and presence of various stickers. The top panels show scattering intensities as a function of q, which are signals from nascent LPS aggregates (micelles or vesicles) at different concentrations, in the absence or presence of the four types of stickers.

FIG. 5B shows the d-spacing measurement of lipid A in the presence of the various stickers. The table summarizes the d-spacing distance under each condition.

FIG. 5C shows the simple model representing the packing density of lipid A in the presence of either sticker-M or sticker-E.

FIG. 6A shows the binding specificity among LPS, stickers and TLR4/MD2 complex, wherein FIG. 6A shows the increasing amounts of two kinds of stickers on LPS-coated plates, which was determined by using an ELISA reader to measure the signal of the SAuNCs adsorbed to the plate at an emission wavelength of about 460 nm.

FIG. 6B shows a noticeable decrease in the binding amount of TLR4/MD2 complex on plates coated with both LPS and stickers in comparison to plates coated with LPS only. The binding specificity of TLR4/MD2 complex was labeled with PE, a dye with an emission wavelength at 594 nm, for measurement.

FIG. 6C shows no significant interaction between stickers and TLR4. The y-axial signal (i.e. amount of LPS-FITC and stickers) was determined by using a calibration curve.

FIG. 7 shows the sticker-M and sticker-E are not immune stimulants. Blood samples were harvested 2 hours after injection and the level of IL-6 in the mouse plasma was measured by the mouse IL-6 ELISA kit.

FIG. 8 shows the Effect of sticker-M on plasma cytokines and CXC chemokines in LPS-challenged mice. Male C57BL/6Narl mice received subcutaneous injections of LPS (0.1 μg) and sticker-M (7.5 μg) into the hind footpad at the indicated time points. Blood samples were harvested at 1 hour and 2 hours after the second treatment for the measurement of TNF-α and other cytokines, respectively.

FIG. 9A-9C shows the effects of sticker-M on measurements of other plasma cytokines and chemokines in LPS-challenged mice. Male C57BL/6Narl mice received subcutaneous injections of LPS (0.1 μg) and sticker-M (7.5 μg) into the hind footpad at the indicated time points. Blood samples were harvested at 2 hours after the second treatment. The P-values were calculated using one-way ANOVA (analysis of variance) with Tukey's multiple comparisons test.

FIG. 10A shows the effect of stickers on the expression of phosphorylated NF-kB p65 (Ser536) in RAW264.7 and bone marrow-derived macrophage (BMDM) of C57BL/6Narl mice in the presence or absence of LPS, respectively. The top image: RAW264.7 cells were stimulated with LPS (20 ng/mL) and harvested at the indicated time points. The middle image: RAW264.7 cells were stimulated with various concentrations of LPS for 30 minutes. The bottom image: LPS-stimulated RAW264.7 cells treated with two kinds of stickers for 30 minutes showed lower expression of the phosphorylated NF-kB p65 (Ser536) protein from whole-cell lysate.

FIG. 10B shows BMDM cells treated with stickers in the presence or absence of 20 ng/mL LPS for 30 min, and the phosphorylated NF-κB p65 (Ser536) expression was detected by western blotting. The relative densities of pSer-NF-κB-p65 protein bands were normalized (3-actin (i.e., loading control), then the calculated fold changes relative to LPS was shown below the blot.

FIG. 11 shows the survival rates of mice with LPS-induced sepsis (25 mg/kg BW) subjected to the treatments with the two kinds of stickers (75 mg/kg BW). The dash line represented the half percentage survival. M and E indicate the sticker-M and sticker-E, respectively.

### DETAILED DESCRIPTION OF THE PREFERRED EMBODIMENTS

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range.

As used herein, the terms “nanocluster” refers to particles with diameters smaller than 2 nm or composed of less than 100 atoms.

The term “gold nanoparticles” refers to spherical gold particles with diameters ranging from larger than 2 nm to 100 nm.

Dendrimers are repetitively branched molecules. A dendrimer is typically symmetric around the core, and often adopts a spherical three-dimensional morphology. Dendrimers are also classified by generation, which refers to the number of repeated branching cycles that are performed during its synthesis. For example if a dendrimer is made by convergent synthesis, and the branching reactions are performed onto the core molecule three times, the resulting dendrimer is considered a third generation dendrimer. Dendrimers are identified by a generation number (Gn) and each complete synthesis reaction results in a new dendrimer generation. Each successive generation results in a dendrimer roughly twice the molecular weight of the previous generation. The first, the second, and the third generation dendrimers are designated as generation 1 (G1), generation 2 (G2) and generation 3 (G3) dendrimers, respectively. Dendrimer-entrapped gold nanoparticles are well-known in the art. For example, the present invention provides for G4 dendrimers.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

HED may vary, depending on other factors such as the route of administration.

Abbreviations: CR: Carbapenem-resistant; AB: Acinetobacter baumannii; EC: Escherichia coli; KP: Klebsiella pneumoniae; PA: Pseudomonas aeruginosa.

### Examples

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Materials

The G₄NH₂ dendrimer, G₄OH dendrimer, HAuCl₄, and LPS (Escherichia coli 0111:B4) were purchased from Sigma, Inc. (San Diego, Calif., USA); an MWCO membrane filter was purchased from Millipore (PES membrane); WST-1 was obtained from Dojindo Laboratories (Kumamoto, Japan). Anion exchange resin was purchased from (Merck, Fractogel® EMD TMAE Hicap).

### Synthesis of Sticker-H, Sticker-A, Sticker-M, and Sticker-E

The sub-nanometer gold nanoclusters (SAuNCs), including sticker-A and sticker-H, were synthesized according to a previously published method. First, HAuCl₄ and HAuBr4 (Sigma-Aldrich, 200 μL, 30 μmol, 150 mM) were added into 20 mL of deionized water containing the G₄NH₂ (Aldrich, 94.9 μL, 5 μmol, 20 wt % methanol solution) and the G₄OH (Aldrich, 75.4 μL, 0.5 μmol, 10 wt % methanol solution), respectively. The G₄NH₂ and HAuCl₄ mixed solution was incubated at 4° C. overnight before being irradiated with microwaves (CEM, Discover LabMate System, 300 W/120° C. for 30 minutes). After reduction, the precipitations and SAuNCs were filtered through a 3 KDa MWCO PES membrane filter (Millipore, Amicon Ultra), and the extra anion, either AuCl4⁻ or AuBr4⁻, was removed by an anionic exchange chromatograph to obtain the purified sticker-A and sticker-H from G₄NH₂ and G₄OH encapsulation, respectively. Then, the internal tertiary amine groups and the surface amine groups of the dendrimer-encapsulated SAuNCs (i.e., sticker-A, 63.6 mg, 4 μmol) were reacted with methyl iodide and ethyl iodide in dichloromethane/N,N′-dimethyl formamide/H2O (1 mL/1 mL/0.1 mL) at room temperature or 37° C. overnight, respectively. Each reaction mixture was extracted by dichloromethane 3 times and then lyophilized to derive two yellow gel-like compounds (i.e., sticker-M and sticker-E). As validation of this decoration, the ¹H NMR spectra showed a main peak at 2.5 ppm˜4 ppm from the methyl and ethyl groups when compared to the sticker-A. All samples were dissolved in D20 as a solvent for measurement.

### High-Resolution Transmission Electron Microscope (HRTEM) Imaging of SAuNCs.

Each sample was mounted on a lacey carbon film. The grid was dried prior to transmission electron microscopy measurements (JEOL JEM-3000F, Japan) at 300 kV.

### Small Angle X-Ray Scattering (SAXS) and Grazing-Incidence Wide-Angle X-Ray Scattering (GIWAXS) Measurements

SAXS data for the LPS solutions were acquired at the 23A SWAXS workstation of the National Synchrotron Radiation Research Center (NSRRC, Taiwan). All the SAXS measurements were formed by using a beam of 15.0 keV (wavelength λ=0.8267 Å) and a sample-to-detector distance of 3060 mm. SAXS data were collected on a pixel detector Dectirs-Pilatus 1M detector with an active area of 169×179 mm² and a detector pixel solution of 172 μm. The scattering wavelength q=4πλ⁻¹ sin θ, defined by the scattering θ and λ, was calibrated with a standard sample of silver behenate. To minimize radiation damage, each 2.5-mm sample solution with thin (12 μm) kapton windows (4 mm in diameter) was gently rocked within an area of 1.5×1.5 mm² to avoid prolonged spot exposure (ca. 0.5 mm in beam diameter) of the sample solution and measured at room temperature. SAXS data were subtracted with buffer scattering measured under an identical environment as that used for the LPS sample solutions; the data were then corrected for incoming flux, sample thickness, and the electronic noise of the detector, as detailed in a previous report. Note that the LPS-to-SAuNCs ratio was kept at 50 (w/w) for each SAXS measurement. GIWAXS measurements were also acquired at the BL23A endstation. Film samples for the GIWAXS measurements were prepared by drop casting on silicon wafer. With a 15 keV (wavelength λ=0.8267 Å) beam, a sample-to-detector distance of 132 mm, and an incident angle of 0.2°, GIWAXS data were collected using a CMOS flat panel X-ray detector C 9728DK (52.8 mm square). The forward scattering intensity (i.e., I(0)) and radius of gyration (Rg) were obtained by Guinier analysis: I(q)=I(0)exp(−R_{g}² q²/3)³, which can be fitted to a linear plot of ln(I) as a function of the square of measured scattering intensity (i.e., q²) (see FIG. 6A, the middle row). The Rg is the average root-mean-square distance to the center of density in the molecule weighted by the scattering length density, and the forward scattering I(0) is proportional to the molecular weight and concentration of LPS micelles or vesicles. The similar Rg values indicated that the size of the LPS aggregates did not change with decreasing concentration.

### A Binding Assay for the Detection of LPS/Sticker Complexes and the TLR4/MD2 Complex

96-well high binding immunoassay plates (Costar 3991, Corning Inc.) were coated with the fixed concentration of LPS 30 μg/ml in 0.1M Na₂CO₃ buffer containing 0.02M EDTA and heated at 37° C. for 200 minutes. After that, the coating plates were washed with deionized water and dried for 16 hours. Then the plates were blocked with 1% BSA in PBS at 37° C. for 30 minutes and washed with 0.1% BSA in PBS. For the detection of binding of two kinds of gold stickers to LPS, the increasing concentrations of either stickers-M or sticker-E were added to the LPS-coated plate. For the detection of inhibition binding of TLR-4 to LPS in the presence of gold stickers, the plate were firstly coated LPS and secondly coated the fixed concentration of either sticker-M or sticker-E, and then the increasing concentrations of TLR-4 conjugated PE (Biolegend) were added to the LPS-stickers-coated plate and read at an emission wavelength at 594 nm. Again, the interaction between gold stickers and TLR4 was determined by commercial mouse Toll-like receptor 4 ELISA kit plate (catalog No: MBS765112, MyBioSource, Inc.). The increasing concentrations of FITC-labeled LPS (Escherichia coli 0111:B4, Sigma) and two kinds of gold stickers were added to the commercial TLR4-coated plate. Y-axial was determined by calibration curves to count the amount of LPS-FITC and stickers. Gold stickers, TLR4-PE, and LPS-FITC were added to each of the indicated coated wells and incubated at 37° C. for 60 minutes. Then the plates were washed with 0.1% BSA in PBS three times. Finally, 100 μl of deionized water was added in each coated well and an ELISA reader (SpectraMax M2, Moleculardevices Inc.) was used to output the fluorescence (gold stickers Ex/Em 390 nm/460 nm, PE Ex/Em 496 nm/594 nm, FITC 496 nm/540 nm).

### Animals

Male 8- to 12-week-old C57BL/6Narl mice were obtained from the National Laboratory Animal Center (Taipei, Taiwan). All the mice were housed under specific pathogen-free conditions with moderate humidity and temperature at the Laboratory Animal Center of the National Health Research Institutes (NHRI). All animal experimental procedures followed published guidelines approved by the NHRI's Institutional Animal Care and Use Committee.

### LPS Treatment

To reveal the interaction of SAuNCs and LPS in vivo, C57BL/6Narl mice received subcutaneous (s.c) injections into the hind footpad of either a single dose of 0.1 μg (50 μL of 2 μg/ml, 4 mg/kg body weight) LPS (Escherichia coli O111:B4, Sigma, Saint Louis, Mo., USA) or a single dose of 7.5 μg SAuNCs in 50 μL PBS. Both the SAuNCs solution and LPS solution were sterilized though syringe filters with a 0.22 μm pore size hydrophilic polyethersulfone (PES) membrane to minimize any contamination. In the protection group, mice were injected with a single dose of LPS 20 minutes after receiving a single injection of SAuNCs. In the treatment group, mice were first injected with LPS and then, after 20 minutes, injected with a single dose of SAuNCs. At two hours after the indicated second injection, blood samples were collected via cardiac puncture following inhalation euthanasia with an isoflurane overdose. For the TNF-α assay, blood samples were drawn at 1 hour after the first injection. In the experimental LPS-induced sepsis model, male C57BL/6Narl mice (average body weight 24.9±1.3 g) were injected intraperitoneally (i.p.) with two kinds of SAuNCs (75 mg/kg body weight) in 100 μL PBS by using a 29 gauge needle at 30-minute intervals before and after receiving a lethal dose 25 mg/kg LPS injection (n=10 per group). Mice were observed at different intervals up to 1 week.

### Plasma

For the initial immunological responses study, a plasma multiplex cytokine assay was performed with a Bio-plex pro mouse 23-plex assay (Bio-rad) by Luminex Bio-Plex 200 system following the manufacturer's instructions. Plasma TNF-α was measured using the mouse TNF-α immunoassay kit (Biolegend) according to the manufacturer's instructions.

### Isolation of Murine Bone Marrow-Derived Macrophages (BMDM)

Bone-marrow cells were isolated from the femurs and tibias of male C57BL/6J mice and the red blood cells were lysed. Bone marrow progenitor cells (5×10⁶ per well) were maintained in complete RPMI-1640 medium with 10% heat-inactivated FBS, 100 U/ml penicillin, 100 μg/ml streptomycin, 100 μM, 2-mercaptoethanol, and 10 ng/ml macrophage colony-stimulating factor (M-CSF, Peprotech) in 6-well plates for 7 days. Before treatment, non-adherent cells were eliminated by removing culture medium. BMDM with unstimulated or stimulated with LPS (20 ng/ml) were treated sticker-M for 30 minutes. BMDM were harvested for the analysis of western bolt.

### Western Blot

Murine raw 264.7 macrophage cells were seeded at 1×10⁶ cell density in a 6 cm Petri dish for 48 hours to grow 90% confluence and maintained in complete RPMI-1640 medium supplemented with 10% heat-inactivated FBS, 100 U/ml penicillin, and 100 μg/ml streptomycin. Raw 264.7 cells were harvested at the indicated time and lysed by RIPA buffer (Sigma) with halt protease and phosphatase inhibitor cocktail (Thermo Scientific). Whole-cell lysates were separated by electrophoresis using 8% SDS-PAGE gels, transferred to PVDF membranes (Millipore), blocked with 5% BSA in TBST for 1 hour, and immunoblotted overnight at 4° C. with primary antibodies, including phosphor-Ser536 NF-κB p65 (Cell Signaling) and beta-actin as loading control. Membranes were exposed to HRP substrate (Millipore) and visualized specific proteins on Amersham Imager 600 (GE Healthcare Life Sciences).

### Pharmacokinetic Study

Male C57BL/6Narl mice were given two intraperitoneal (i.p.) injections of SAuNCs (75 mg/kg body weight, in 100 μL steriled PBS) with a 60-minute interval using a 29 gauge needle. Blood samples were drawn at 0 minute (baseline), 15 minutes, 30 minutes, 60 minutes, 75 minutes, 90 minutes, 105 minutes, 120 minutes, 3 hours, 4 hours, 5 hours, 6 hours, 8 hours, 12 hours, 24 hours, and 48 hours after the first injection of SAuNCs. Whole blood samples were collected in purple-top blood collection tubes containing EDTA as an anticoagulant and measured for Au-atom content by inductively coupled plasma mass spectrometry (ICP-MS). Pharmacokinetic parameters were calculated using the Excel software and included the half-life (t_{1/2}), time of maximal plasma concentration (Tₘₐₓ), maximal plasma concentration (Cₘₐₓ), and area under the concentration-time curve (AUCₐₗₗ).

### Statistical Analyses

The GraphPad Prism program (v7.02) was used to conduct statistical analyses. Data for the biologic assays were expressed as mean±standard deviation (SD), and the P-values were calculated using one-way ANOVA (analysis of variance) with Tukey's multiple comparisons test, with P<0.05 considered to indicate a statistically significant test result. In the experimental LPS-induced sepsis model, the survival rate data were plotted using Kaplan-Meier curves and analyzed by the Log-rank (Mantel-Cox) test to compare the SAuNC group with the LPS-alone group.

### Result

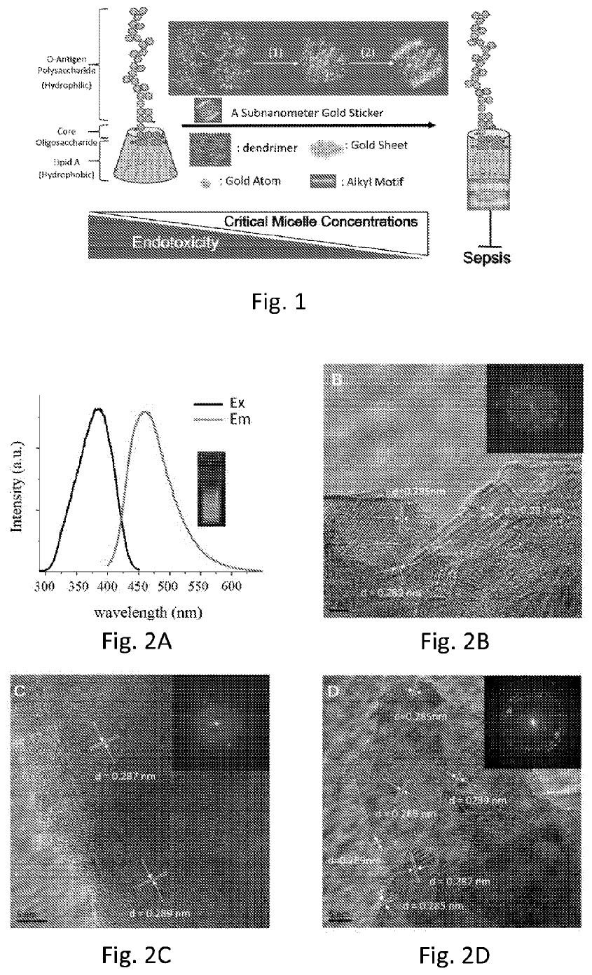

In the present work, we sought to construct an ultra-small gold sticker to block endotoxin activity by compacting of the d-spacing of lipid A (an endotoxicity active site of LPS). By manipulating the intramolecular packing density, the conformation of lipid A domains can be converted from a looser to a denser density, which could, in turn, dramatically influence innate immune recognition. The difference in the d-spacing resulting in looser or denser packing is a matter of only several angstroms. In order to fine-tune such a subtle change, the anti-endotoxin sticker would need to be composed by an adhesive-like motif consisting of soft materials and an ultra-small but hard substrate with a flake-like geometry. Such a sticker-like structure would be expected to influence the d-spacing of lipid A, in addition to increasing the critical micelle concentration (CMC) for the inhibition of LPS non-lamellar aggregation. However, most hard nanometer-scale materials, i.e., inorganic nanoparticles, have stereoscopic geometries with different curvatures that make them unsuitable for use as the substrate of a flake-like sticker. Fortunately, when the size of nanoparticles is shrunk to sub-nanometer ranges, the geometries of such particles can be changed to flake-like geometries. For example, sub-nanometer gold nanoclusters (SAuNCs) with a flake-like geometry have already been established theoretically. We hypothesized that such a flattened face of SAuNCs might easily allow an attached adhesive-like motif to dock with the lipid A domain of LPS by compacting the intramolecular d-spacing of lipid A (FIG. 1), thereby reducing the recognition of TLR4-MD2 complex for the development of endotoxin-induced sepsis.

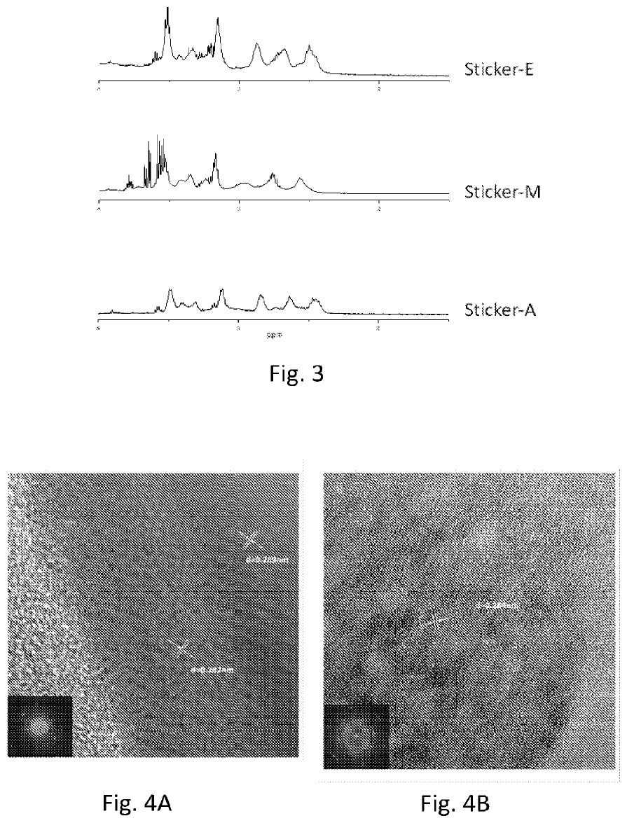

FIG. 2A shows a blue photoluminescence with excitation and emission peaks at ˜390 nm and ˜460 nm, respectively, from the SAuNCs used in this study. While the issue of how to exactly measure the size of SAuNCs poses a big challenge, the emission wavelength of photoluminescence allows for reasonable estimates of how many gold atoms compose a single nanocluster. The maximum value of the emission wavelength appearing at ˜460 nm indicates that the SAuNCs are Au8-dominated nanoclusters (i.e., where Au8 consists of eight gold atoms). The synthetic protocol based on the formation of a dendrimer-encapsulated SAuNCs has been published elsewhere, with mass measurements having demonstrated the Au8-dominated nanocluster within one dendrimer as a main product. Otherwise, the entire size of dendrimer-encapsulated SAuNCs has been reported to be only approximately 2 nm due to the fact that the embedding of gold atoms can cause an irreversible back-folding of the exterior amines of dendrimers, resulting in the conformation contraction of the dendrimers. It should be emphasized that the conformational contraction of dendrimers can easily happen while adjusting various parameters, including pH and solvent polarity and ion strength. The dimension of SAuNCs is estimated to be less than 1 nm, and such nanoclusters are thought to possibly have a flake-like shape. Thus, we further used a high-resolution transmission electron microscopy (HRTEM) to study the shape of the SAuNCs. It is very surprising that the nanoclusters could form stacks in a layer-by-layer manner (FIG. 2B to 2D) on copper grids, and some domains showed a well-ordered alignment (FIG. 2C), from which it can be deduced that these SAuNCs can self-assemble spontaneously into thin-films. Furthermore, we found that the neighbor distance of gold atoms ranged from 0.285˜0.289 nm (as indicated by the white arrows shown in FIG. 2B to 2D), which is very close to the theoretical value (i.e., 0.288 nm) of the nearest neighbor spacing. The atomic resolution provides direct evidence to confirm that the alignment was consistent with that of dendrimer-encapsulated SAuNCs rather than gold nanoparticles. That is, the alignment was unlike that of the superstructure of thiol-capped SAuNCs formed from the coalescence of gold atoms, which results in the formation and alignment of gold nanoparticles. The layer-by-layer stacking of the SAuNCs used in this study also illustrated that the capping molecule (i.e., the deformed dendrimer) might avoid the coalescence of gold atoms, as well as assist in the self-assembly of the SAuNCs. As a result, the observation of thin-films also can explain that the geometry of SAuNCs consists of a flake-like structure that can allow layer-by-layer alignment. Based on this observation, the SAuNCs were then decorated with two kinds of alkyl motifs, methyl and ethyl groups, that were used as adhesives and resulted in the sticker-M and sticker-E, respectively. The detailed synthesis and characterization of these stickers are described in the supplemental text and shown in FIG. 3 to FIG. 4B.

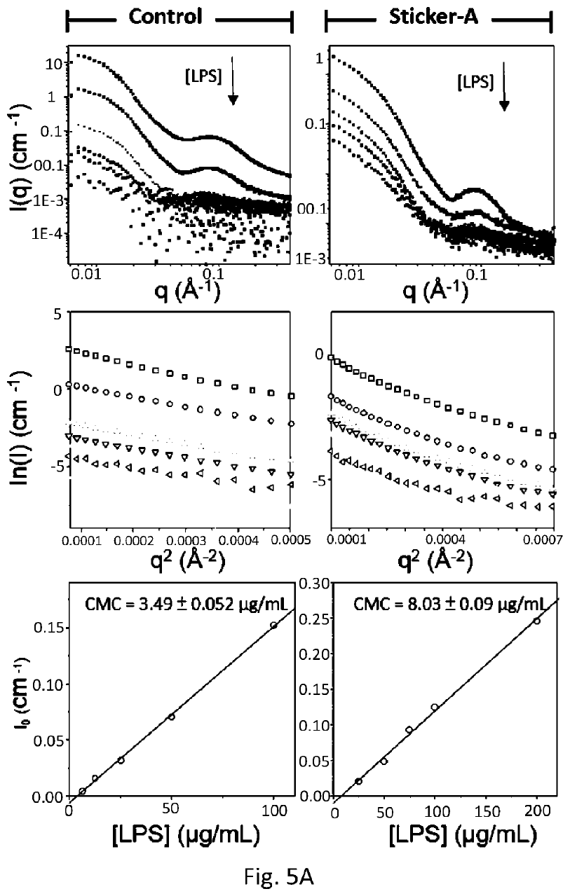

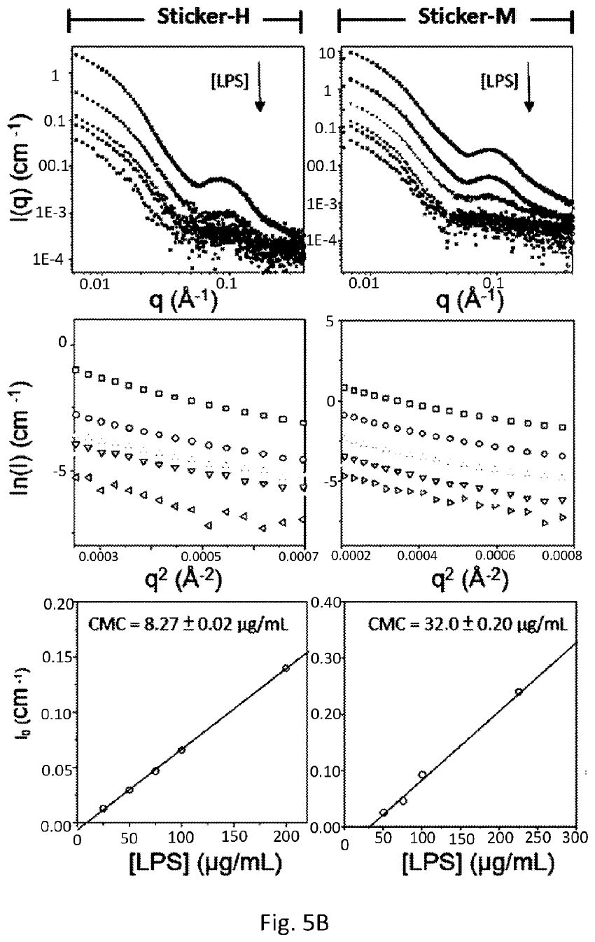

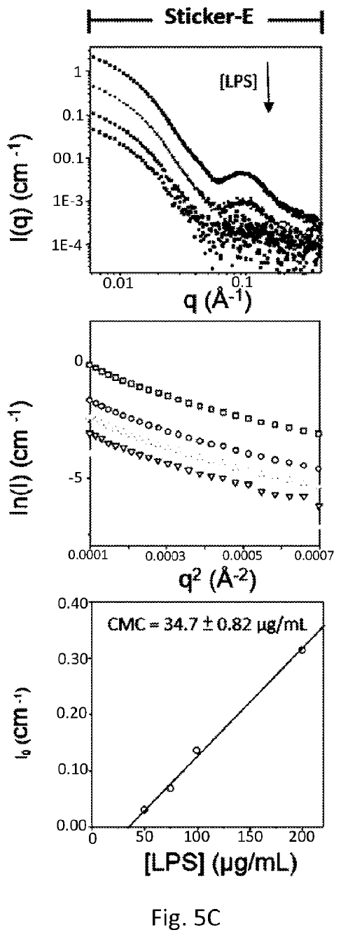

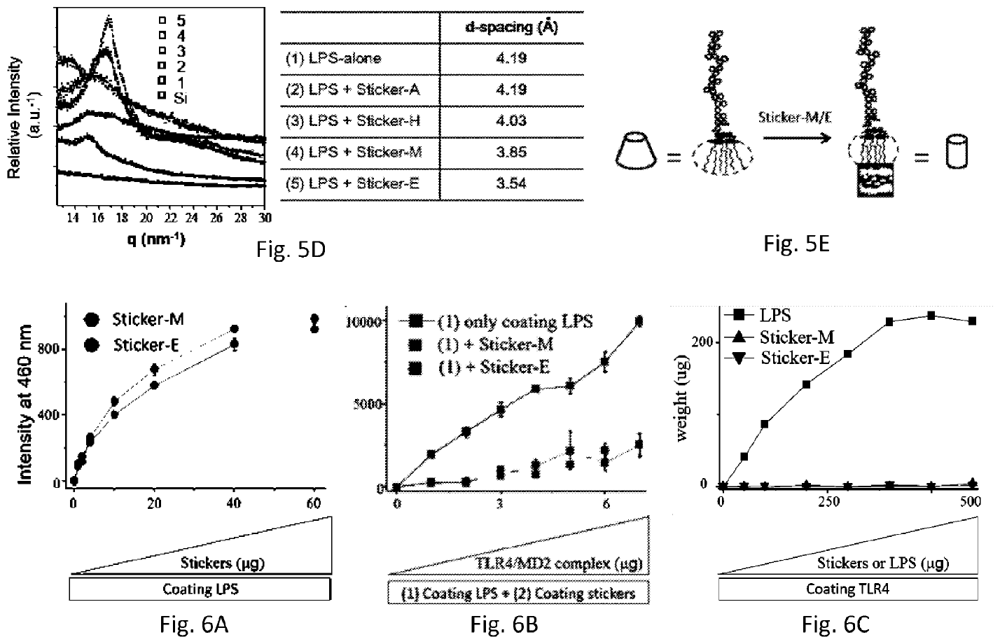

Next, it is very interesting to determine whether the CMC of LPS can be influenced in the presence of either sticker-M or sticker-E, resulting in the inhibition of LPS aggregation. For comparison, we also prepared other hydrophilic and hydrophobic SAuNCs (sticker-A and sticker-H, respectively) that did not, however, include the decorative alkyl-motifs that have been validated to cause LPS aggregates; that is, the CMC of the LPS could not be affected by them. FIG. 5A (the top row) shows an intense signal around 0.1 A⁻¹ that determined the aggregate formation of LPS in the absence and presence of various stickers by small angle X-ray scattering (SAXS). According to Guinier analysis, the forward scattering intensity (I₀) was obtained by fitting a linear plot of ln(I) as a function of the square of the measured scattering intensity (q²) (FIG. 5A, the middle row). Since the I₀ showed a good linearity in relation to the low solution concentrations (FIG. 5A, the bottom row), the CMC values of the LPS in each condition could then be calculated via linear extrapolation. As expected, FIG. 5A (the fourth/fifth column) shows that the CMC value of the LPS in the presence of either sticker-M or sticker-E was significantly increased (by a ten-fold magnitude) over that of the LPS alone (the first column). These effects might be attributed to the interactions of the methyl and ethyl motifs on the SAuNCs with lipid A, which could have resulted in the inhibition of the self-assembly process of LPS. Thus, the measurement of the d-spacing of lipid A by using grazing-incidence wide-angle X-ray scattering (GIWAXS) was shown in FIG. 5B (the left side). The results found that both sticker-M and sticker-E caused an observable change of scattering vector (q), changing it from 14.96 nm⁻¹ to 16.32 nm⁻¹ and 17.72 nm⁻¹, respectively. The values of the d-spacing (2π/q) for lipid A in each condition were then calculated, and listed in FIG. 5B (the right side). The d-spacing values are about in distribution from 4.19 Å to 3.54 Å. It is notable that only sticker-H/M/E resulted in compacting of the d-spacing of lipid A in comparison to the d-spacing for lipid A seen with LPS alone; that is, sticker-A resulted in no compacting. These results indicated that stickers with hydrophobic moieties, especially those with only methyl and ethyl motifs, could reduce the intramolecular d-spacing of each individual LPS molecule, resulting in more dense packing densities (FIG. 5C), in addition to increasing the CMC, and that might be protecting against sepsis.

Besides the stickers that can directly dock the lipid A of LPS, as mentioned above, it is required to evaluate whether sticker-M and sticker-E can or cannot act as an antagonist to bind with TLR4/MD2 complex as well. As expected, we found that both sticker-M and sticker-E can bind to LPS and present a dose-dependent response (FIG. 6A), but cannot associate with TLR4 (FIG. 6C). As a result, the conclusion that our stickers behaved as TLR4 antagonists can be ruled out. More importantly, the association of LPS and the TLR4/MD2 complex is dramatically reduced in the presence of sticker-M and sticker-E in comparison to their association in the presence of LPS alone (FIG. 6B). This observation suggested that sticker-M and sticker-E might only engage with the lipid A of LPS to interrupt the interactions between LPS and various proteins. As such, based on our strategy, the stickers might become an effective inhibitor for anti-endotoxin.

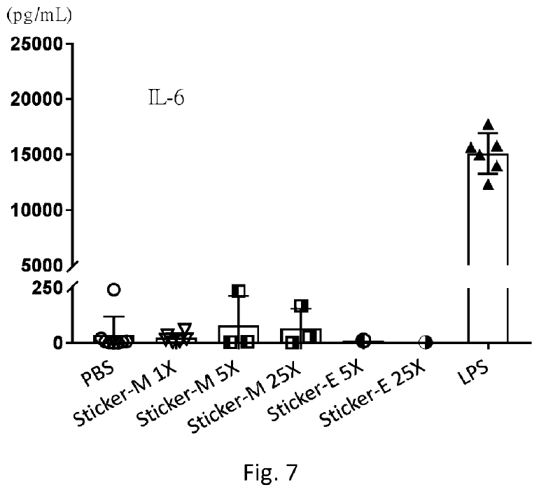

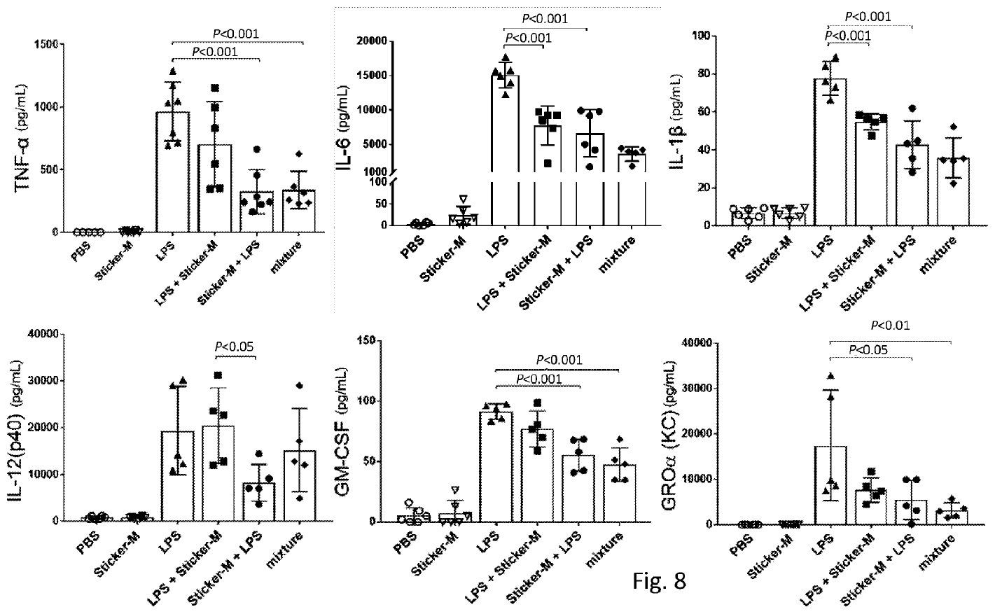

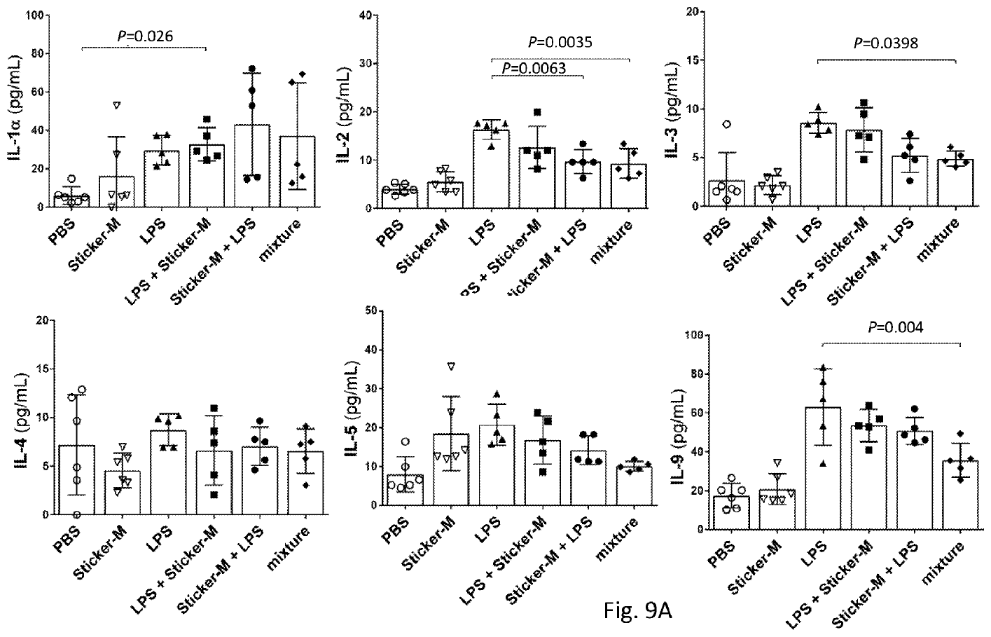

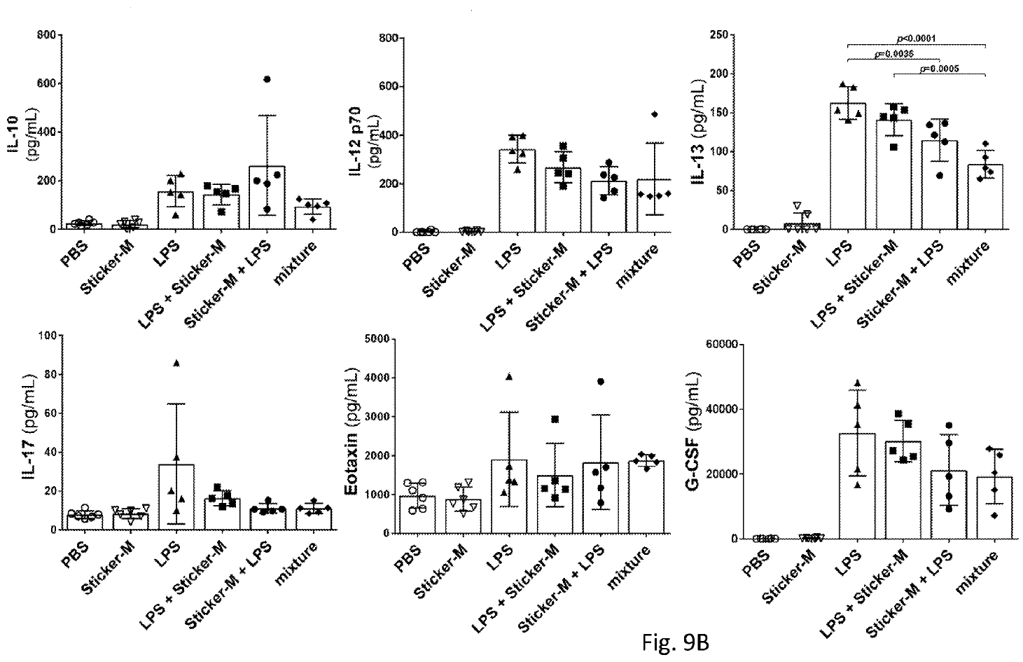

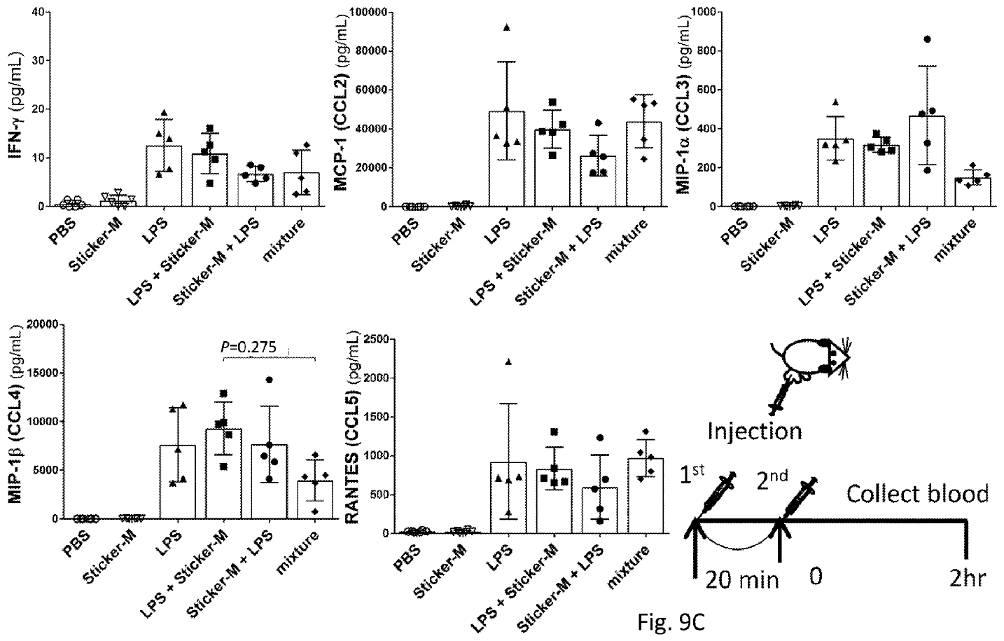

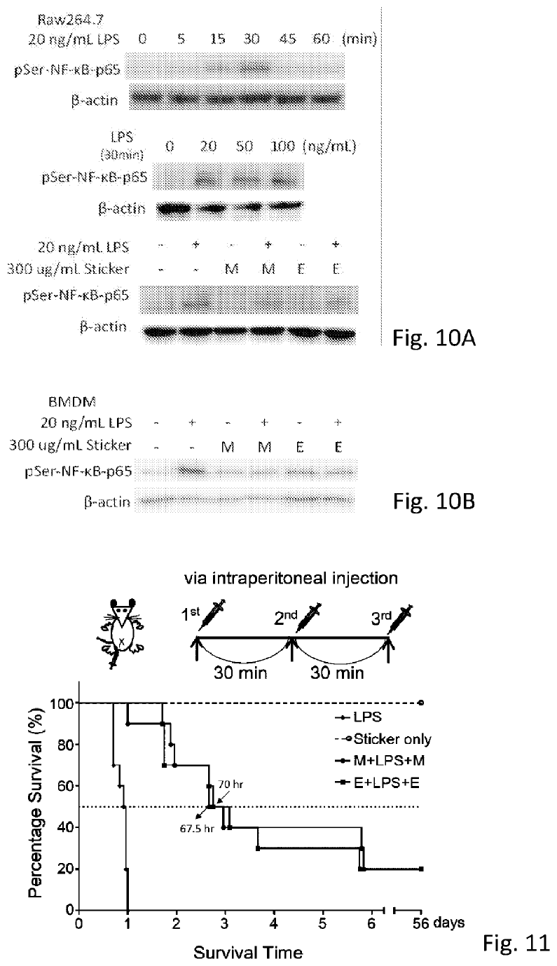

The hypothesis that the compacting of lipid A by the ultra-small gold stickers can protect mice from LPS-induced inflammation needs to be validated. Note that neither sticker-M nor sticker-E alone is regarded as an immune stimulant (FIG. 7). For simplification, only sticker-M was investigated by a detailed study of the induction of key pro-inflammatory cytokines and chemokines in LPS-challenged mice. Regardless of the pre-treatment or post-treatment of sticker-M injections, the inhibition of LPS-induced cytokine/chemokine induction was comparable to the injections of the premix of LPS and sticker-M (FIG. 8 and FIG. 9A-9C, mixture column, labeled as mixture). For example, the concentrations of pro-inflammatory NF-κB-dependent cytokines, including plasma TNF-α, IL-6 and IL-1β, in the LPS-challenged mice were significantly reduced. Again, the plasma immunostimulatory IL-12p70 levels were not changed, whereas the plasma IL-12p40 levels resulting from pre-treatment with sticker-M were lower than those resulting from post-treatment with sticker-M. IL-12p40 plays an immunoregulatory role as the bridge between innate and adaptive defense immunity. Meanwhile, the levels of both plasma GM-CSF and GROα (KC), which are secreted by innate immune cells in response to LPS challenge,¹ were significantly decreased in the mice that underwent sticker-M injection. Other production of plasma cytokines and CXC chemokines profiles (FIG. 9A-9C) as well as expression of phosphorylated NF-κB (FIG. 10A and FIG. 10B) from RAW264.7 and bone marrow-derived macrophage (BMDM) also showed a noticeable decrease. Taken together, the results suggest that the ultra-small gold sticker-M might affect lipid A function and thereby lead to a decrease in the cell cytotoxicity of endotoxin during earlier events after LPS injection.

TABLE 1
The pharmacokinetics of two kinds of ultra-small gold stickers

| | Sticker-M | Sticker-E |
|---|---|---|
| t_{1/2} (hr) | 17.4 | 15.4 |
| T_{Max} (hr) | 1.3 | 1.3 |
| C_{Max} (ng/ml) | 198.6 | 427.6 |
| AUCₐₗₗ (hr*ng/ml) | 2912.2 | 5794.5 |

Pharmacokinetics study shows that the 17.4-hour half-life of sticker-M was slightly longer than that of sticker-E (15.4 hour) as shown in Table 1. The preventive effect in LPS-induced septic mice of using sticker-M and sticker-E was validated (FIG. 11).

TABLE 2
MIC (minimum inhibitory concentration) test

| | Stickers | | |
|---|---|---|---|
| Strains | M | E | P |
| CRAB | >2048 | >2048 | >2048 |
| CREC | >2048 | >2048 | >2048 |
| CRKP | >2048 | >2048 | >2048 |
| CRPA | >2048 | >2048 | >2048 |

Method: broth dilution (Mueller-Hinton II broth, Unit: μg/mL); Ref. Clinical and Laboratory Standards Institute. 2015. M100-S25. Performance standards for antimicrobial susceptibility testing, 25th informational supplement. Clinical and Laboratory Standards Institute, Wayne, PA. CLSI, Wayne, PA, USA, 2015.

Since our stickers could not kill gram-negative bacteria (Table 2), the experimental sepsis mimicked the release of endotoxin debris after bacterial death. While the median survival time of LPS-induced septic mice without sticker injections was 22.5 hours, the median survival time of LPS-induced septic mice pre-treated with sticker-M and sticker-E was 67.5 hours and 70 hours, respectively. Two kinds of stickers (i.e. sticker-M and sticker-E) significantly prolonged the survival time with a 3-fold increase in LPS-induced septic mice. The survival time of the LPS-induced septic mice injected with sticker-E was slightly longer than that of the LPS-induced septic mice injected with sticker-M. It is speculated that this greater improvement was due to the fact that the ethyl motifs could adhere more strongly to lipid A than the methyl motifs due to an intermolecular van der Waals force. Collectively, the ultra-small gold with decorated methyl and ethyl motifs might potentially function as an anti-endotoxin sticker.

In summary, we present herein a sub-nanometer gold sticker that can efficiently block endotoxin activity as a means of counteracting sepsis. The ultra-small sticker consists of a gold nanocluster that serves as a flake-like substrate and a coating of short alkyl motifs that act as an adhesive for docking with LPS, a dangerous debris of gram-negative bacteria, through targeting lipid A and compacting its intramolecular hydrocarbon chain-chain distance (d-spacing). In biological relevance, the induction of key pro-inflammatory NF-κB-dependent cytokines, including plasma tumor necrosis factor-alpha (TNF-α), IL-6 and IL-1β and chemokines in LPS-challenged mice showed a noticeable decrease. Not only that, the treatment of anti-endotoxin stickers could significantly prolong the survival time in LPS-induced septic mice. The injection of anti-endotoxin stickers might constitute a potential therapeutic strategy for the early prevention of sepsis caused by gram-negative bacterial infection, effectively protecting the patients from systemic inflammatory response syndrome (SIRS), septic shock, and sepsis-induced lethality.

1. A sub-nanometer gold sticker, wherein the sub-nanometer gold sticker is comprising gold nanoparticles encapsulated within a polyamidoamine (PAMAM) dendrimer and a coating of alkyl motif.

2. The sub-nanometer gold sticker of claim 1, wherein the gold nanoparticles are encapsulated within a dendrimer to form a gold nanoparticle-dendrimer complex.

3. The sub-nanometer gold sticker of claim 2, wherein the shape of the gold nanoparticle-dendrimer complex is about flake-like structure.

4. The sub-nanometer gold sticker of claim 1, wherein the PAMAM dendrimer is GₙNH₂ dendrimer or GₙOH dendrimer, wherein n is 0 to 4.

5. The sub-nanometer gold sticker of claim 4, wherein the PAMAM dendrimer is a generation 4 (G4) dendrimer.

6. The sub-nanometer gold sticker of claim 5, wherein the PAMAM dendrimer is G₄NH₂ dendrimer or G₄OH dendrimer.

7. The sub-nanometer gold sticker of claim 1, wherein the alky motif includes methyl groups or ethyl groups.

8. A method of blocking endotoxin activity, comprising steps of administering to a mammal in need thereof an effective amount of sub-nanometer stickers to compact the intramolecular hydrocarbon chain-chain distance (d-spacing) of lipid A of LPS.

9. The method of blocking endotoxin activity of claim 8, wherein the d-spacing values of lipid A is decreasing from 4.19 Å to 3.54 Å.

10. The method of blocking endotoxin activity of claim 9, wherein the d-spacing values of lipid A is decreasing from 4.19 Å to 3.85 Å.

11. A method of suppressing pro-inflammatory cytokines, comprising steps of administering to a LPS-infected mammal in need thereof an effective amount of sub-nanometer stickers.

12. The method of suppressing pro-inflammatory cytokines of claim 11, wherein the effective amount of sub-nanometer stickers is dependent on the molar ratio of sub-nanometer stickers to LPS.

13. The method of suppressing pro-inflammatory cytokines of claim 11, wherein the molar ratio of stickers to LPS is 1:2.

14. The method of suppressing pro-inflammatory cytokines of claim 11, the effective amount of sub-nanometer stickers is about 50˜100 mg/kg body weight.

15. The method of suppressing pro-inflammatory cytokines of claim 11, wherein the pro-inflammatory cytokines include NF-κB, TNF-α, IL-6, CXC chemokines profiles, IL-12p40, GM-CSF or GRPα (KC).

16. A method of protecting against endotoxin-induced sepsis, comprising steps of administering to a mammal in need thereof an effective amount of sub-nanometer stickers to increase critical micelle concentration for the inhibition of LPS non-lamellar aggregation.
